(19) United States
(12) Patent Application Publication
(10) Pub. No.: US 2025/0164415 A1
(43) Pub. Date: May 22, 2025

(54) X-RAY IMAGING APPARATUS

(71) Applicants: RAYENCE Co., Ltd., Gyeonggi-do (KR); VATECH EWOO Holdings Co., Ltd., Gyeonggi-do (KR); Qpix solutions Inc., Morrisville, NC (US)

(72) Inventors: Seungman YUN, Morrisville, NC (US); Changhyeuk KIM, Morrisville, NC (US); Byeongnyeon KIM, Gyeonggi-do (KR); Jongbo YOON, Gyeonggi-do (KR)

(73) Assignees: RAYENCE Co., Ltd., Gyeonggi-do (KR); VATECH EWOO Holdings Co., Ltd., Gyeonggi-do (KR); Qpix solutions Inc., Morrisville, NC (US)

(21) Appl. No.: 18/952,959

(22) Filed: Nov. 19, 2024

Related U.S. Application Data
(60) Provisional application No. 63/600,898, filed on Nov. 20, 2023.

(51) Int. Cl. G01N 23/04

(52) U.S. Cl. CPC G01N 23/04; G01N 2223/1016; G01N 2223/301; G01N 2223/308; G01N 2223/321

(57) ABSTRACT

Proposed is a C-arm X-ray imaging apparatus in which an arm part connecting a main body part and a C-arm part and a C-arm supporter are lightweight and simple, and have stable operating and stationary postures. The apparatus includes a main body part including a movable mobile base, an arm part connected to the main body part through a first axis and capable of rotating up, down, left and right based on the first axis, a C-arm supporter connected to the arm part through a second axis and capable of rotating left and right based on the second axis, and a C-arm part including a C-shaped arm connected to the C-arm supporter through a third axis and capable of rotating, and an X-ray generator and an X-ray detector respectively disposed at opposite ends of the C-shaped arm, wherein the first axis and the second axis remain parallel to each other with respect to the up-down, left-right rotation of the arm part.

### CROSS REFERENCE TO RELATED APPLICATION

This application claims the benefit of the filing date of U.S. Patent Provisional Application No. 63/600,898, filed Nov. 20, 2023, as Attorney Docket No.: 810.0160prov, the teaches of which are incorporated herein their entirety by reference.

### BACKGROUND

### Technical Field

The present disclosure relates to an X-ray imaging apparatus in which an X-ray generator and an X-ray detector are respectively disposed at opposite ends of a C-shaped arm.

### Description of the Related Art

An X-ray machine is a device that emits X-rays toward a subject, obtains an X-ray image from the X-rays that travel through the subject to display the internal structure of the subject.

An example of the X-ray machine is an X-ray fluoroscopy apparatus, commonly called a C-arm, which is used in operating rooms, emergency rooms, or during other clinical procedures. As an example, the C-arm X-ray machine consists of a mobile base, a multi-joint arm connected to the mobile base, and a C-shaped arm connected to the multi-joint arm. Additionally, an X-ray generator and an X-ray detector are respectively installed at opposite ends of the C-shaped arm to capture an X-ray fluoroscopy image of an object placed therebetween.

The C-arm X-ray machine is mainly used to acquire two-dimensional X-ray fluoroscopy images of a subject. Meanwhile, in order to check the exact location of a lesion, surgical progress, location of a surgical instrument, and degree of fastening during surgery, tomographic images may be required. However, it is difficult to move a patient to an imaging room equipped with a CT imaging system during surgery to obtain tomographic images. A C-arm X-ray machine with a tomosynthesis function, which involves acquiring multiple tomographic images of an object by emitting X-rays from different angles within a limited angular range, can be an alternative.

The problem is that, to obtain a tomographic image of tomosynthesis, the X-ray generator must rotate and move to obtain multiple projection images of a subject over a certain angle range. In a typical C-arm X-ray machine, because an X-ray generator is fixed to one end of a C-shaped arm, the entire C-shaped arm needs to be rotated to obtain multiple projection images in a certain angle range. In the case of a conventional C-arm X-ray machine with a large and complex driving mechanism, the imaging time is long, precise control is difficult, and due to increased load and volume, it is often difficult to use the machine in an operating room or even get the machine into the operating room. To increase the usability of a C-arm X-ray machine, it may be possible to consider increasing the degree of freedom of a multi-joint arm connecting a base and a C-shaped arm. However, the high degree of freedom may actually make it difficult for a user to operate the machine and may reduce the accuracy of the imaging posture.

### DOCUMENT OF RELATED ART

(Patent Document 1) Korean Patent Application Publication No. 10-2016-0071938
(Patent Document 2) Korean Patent Application Publication No. 10-2013-0058633
(Patent Document 3) U.S. Patent Application Publication No. 2020-0085390

### SUMMARY

Accordingly, the present disclosure has been made keeping in mind the above problems occurring in the related art, and the present disclosure is intended to provide an X-ray imaging apparatus that does not expose the movement of a drive system including an X-ray generator during tomosynthesis, that has a limited footprint so as to operate and be moved in a tight space such as an operating room and a treatment/procedure room, and that has an arm part, which connects a main body part and a C-arm part, and a C-arm supporter that are lightweight and simple, yet enable stable operation and control.

In order to achieve the above objective, according to an aspect of the present disclosure, there is provided an X-ray imaging apparatus including: a main body part configured to include a movable mobile base; an arm part connected to the main body part through a first axis and capable of rotating up, down, left and right based on the first axis; a C-arm supporter connected to the arm part through a second axis and capable of rotating left and right based on the second axis; and a C-arm part configured to include a C-shaped arm connected to the C-arm supporter through a third axis and capable of rotating, and an X-ray generator and an X-ray detector respectively disposed at opposite ends of the C-shaped arm, wherein the first axis and the second axis may remain parallel to each other with respect to the up-down, left-right rotation of the arm part.

The arm part may include: upper and lower links parallel to each other; a rear axis member connected to the first axis and to which rear ends of the upper and lower links are hinge-coupled; and a front axis member connected to the second axis and to which front ends of the upper and lower links are hinge-coupled.

The apparatus may further include a gas spring configured to connect the front axis member and the lower link or the rear axis member and the upper link.

The C-arm supporter may further include a slide connection part connected through the third axis, wherein the C-shaped arm may be connected to the slide connection part so as to slide along the slide connection part.

The slide connection part may further include a C-arm fixing knob configured to screw through the slide connection part and secure a position of the C-shaped arm relative to the slide connection part.

The C-shaped arm may include a receiving space provided along a length direction to accommodate the slide connection part, and the slide connection part may include a plurality of rollers rotating along the receiving space within the receiving space.

According to the present disclosure, provided is an X-ray imaging apparatus that does not expose the movement of a drive system including an X-ray generator during tomosynthesis, that has a limited footprint to be able to operate and be moved in a tight space such as an operating room and a treatment/procedure room, and that has an arm part, which connects a main body part and a C-arm part, and a C-arm supporter that are lightweight and simple, yet enable stable operation and control.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a side view of an X-ray imaging apparatus according to an embodiment of the present disclosure with a C-shaped arm extended forward;

FIG. 2 is a side view of the folded state of the X-ray imaging apparatus according to an embodiment of the present disclosure;

FIG. 3 shows a C-arm part and a portion of an arm part of the X-ray imaging apparatus according to an embodiment of the present disclosure;

FIG. 4 is a side view of the internal structure of the arm part of the X-ray imaging apparatus of FIG. 1;

FIG. 5 schematically shows the operating principle of the arm part of the X-ray imaging apparatus according to an embodiment of the present disclosure;

FIG. 6 is a view of the C-arm part according to an embodiment of the present disclosure as seen from the C-shaped arm side;

FIG. 7 shows the cross-section (VII-VII′) profile of the C-shaped arm of FIG. 6; and

FIG. 8 shows the configuration of a C-arm supporter connected to the C-shaped arm of FIG. 6.

### DETAILED DESCRIPTION

Hereinafter, embodiments of the present disclosure will be described in detail with reference to the drawings. The technical idea of the present disclosure may be more clearly understood through the embodiments. The present disclosure is not limited to the embodiments described below.

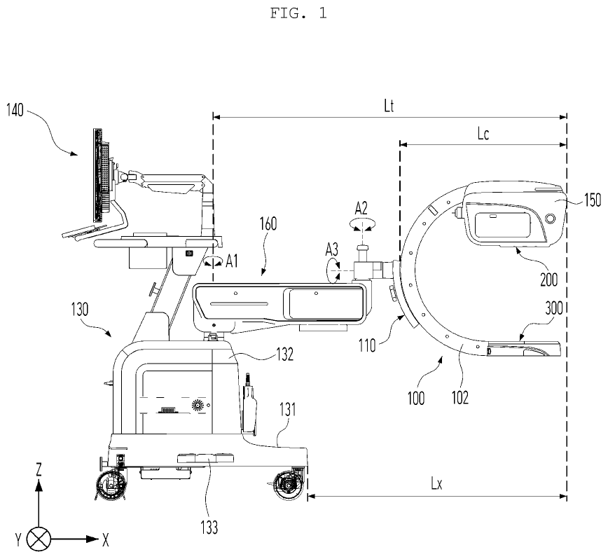

FIG. 1 is a side view of an X-ray imaging apparatus according to an embodiment of the present disclosure with a C-shaped arm extended forward.

In FIG. 1 and the following drawings, the x-axis direction represents the front of the X-ray imaging apparatus, the y-axis direction represents a direction perpendicular to the x-axis on the plane where the X-ray imaging apparatus is placed, and the z-axis direction represents a direction perpendicular to the plane.

The X-ray imaging apparatus according to this embodiment of the present disclosure largely include a main body part 130, a C-arm part 100, and an arm part 160 connecting the main body part 130 and the C-arm part 100. A mobile base 131 is provided at the lower part of the main body part 130 to support the entire load of the apparatus and to be movable. Additionally, the main body part 130 may have a built-in controller 140 such as a console PC responsible for input to the apparatus and image processing. The controller 140 may include a display disposed above the main body part 130 to output an X-ray image, etc.

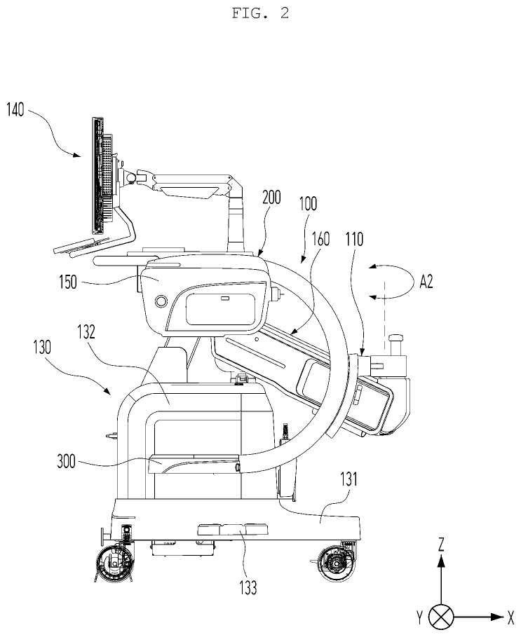

The mobile base 131 may include a plurality of wheels and a frame supporting the wheels for stable movement. In addition, when viewed from above as shown in FIG. 2, in the mobile base 131, the area between the parts supporting the two front wheels of the frame is concavely recessed toward a body 132 of the main body part 130 to reduce interference with the feet of a user moving or operating the apparatus.

The rear end of the arm part 160 is connected to the main body part 130 and the front end of the arm part 160 is connected to the C-arm part 100. To be specific, the rear end of the arm part 160 is connected to the main body part 130 so as to rotate up, down, left, and right within at least a predetermined angle range about a first axis A1 parallel to the z-axis. The front end of the arm part 160 and the C-arm part 100 are connected through a C-arm supporter 110.

The C-arm part 100 includes an X-ray generator 200 (hereinafter referred to as generator), an X-ray detector 300 (hereinafter referred to as detector), and a C-shaped arm 102 with the X-ray generator 200 and the X-ray detector 300 respectively disposed at opposite ends.

The C-arm supporter 110 has one side thereof supporting the C-arm part 100 so as to slide along the C-shaped curved surface of the C-shaped arm 102, and has the other side thereof rotatably connected to the front end of the arm part 160 about a second axis A2. One side of the C-arm supporter 110 connected to the C-arm part 100 may be rotatably connected to the other side of the C-arm supporter 110 connected to the arm part 160 about a third axis A3 perpendicular to the second axis A2.

The body 132 is disposed biased toward the rear from the center of the mobile base 131, and the first axis A1 may be disposed in a position overlapping with the upper surface of the body 132 when viewed from above. This arrangement is advantageous for maintaining the center of gravity within the footprint of the mobile base 131 and allowing the C-arm part 100 to fully extend forward. Such arrangement is also advantageous in reducing the space occupied when the C-arm part 100 and the arm part 160 are folded for storage or movement.

FIG. 2 is a side view of the folded state of the X-ray imaging apparatus according to an embodiment of the present disclosure.

When moving or storing the X-ray imaging apparatus according to the present disclosure, as shown, the arm part 160 and the C-arm part 100 may be transformed into a folded state in which the arm part 160 and the C-arm part 100 are folded to the side of the main body part 130 and are in close contact. At this time, the arm part 160 and the C-arm part 100 rotate about the first axis A1 and the second axis A2, respectively, so that the C-arm part 100 is located on the side of the body 132 of the main body part 130. The C-arm part 100 may be configured to be temporarily fixed to the side of the body 132 by using a magnet or other temporary fixing means provided on the body 132. In this case, the direction in which the arm part 160 and the C-arm part 100 are folded is not limited, and may be configured to be folded on both left and right sides.

This folded posture lowers the center of gravity of the entire apparatus, adding stability during movement and storage, and reduces the extent to which the arm part 160 and the C-arm part 100 protrude out of the mobile base 131, providing the effect of greatly reducing the volume during movement and storage.

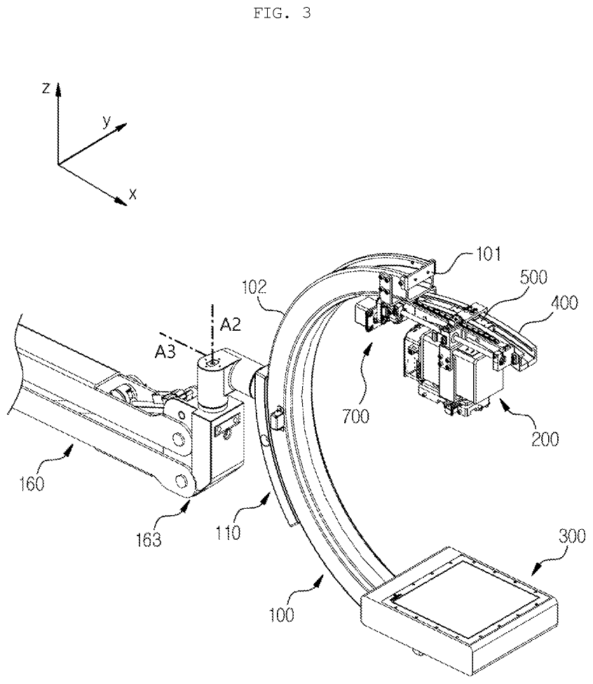

FIG. 3 shows a C-arm part and a portion of an arm part of the X-ray imaging apparatus according to an embodiment of the present disclosure. For convenience, FIG. 3 shows a generator part casing 150 removed from FIG. 1.

The C-shaped arm 102, which constitutes the framework of the C-arm part 100, may slide along a C-shaped bend with respect to the C-arm supporter 110.

In the C-arm part 100, an arcuate guide rail 400 is disposed at one end of the C-shaped arm 102 in the front-back direction of the extension line, the generator 200 is arranged to move in the front-back direction along the arcuate guide rail 400. To this end, the C-arm part 100 includes a driving part 700 that provides power to move the generator 200. The driving part 700 may include a motor. In addition, the driving part 700 may be configured to convert the rotational motion of the motor into linear motion, and transmit the linear motion back to a moving block 500 disposed within the arcuate guide rail 400, so that the linear motion is converted into an arc-shaped trajectory motion of the moving block 500. The generator 200 is fixed to the moving block 500 and moves along the arcuate guide rail 400. At this time, preferably, the generator may move along the guide rail 200 while maintaining an equal distance with respect to the detector. However, the above-described power transmission method is not limited and may be implemented in various ways, such as directly transmitting the power of the drive motor to the motion of an arc-shaped trajectory using a flexible power transmission means such as a chain or timing belt.

The X-ray imaging apparatus according to the present disclosure may acquire multiple projection images in a limited angle range by receiving X-rays that have passed through a subject (not shown) located between the generator 200 and the detector 300 at various angles at the detector 300, and reconstruct tomosynthesis images, which are multiple tomographic images of the subject, from the projection images. Additionally, as the generator 200 moves along the arcuate guide rail 400 connected to one end of the C-shaped arm 102 for tomosynthesis imaging, the mechanical configuration of the C-arm part 100 may be simplified compared to the case where the entire C-arm part 100 moves.

In addition, in FIG. 3, the internal structure of a portion of the front side of the arm part 160 may be seen. As shown, the arm part 160 may be configured to include a pair of links parallel to each other, so that while up-and-down rotation around the A1 axis is possible, even if the angle of the arm part 160 changes, the first axis A1 and the second axis A2 (see FIG. 1) disposed at opposite ends of the arm part 160 may always be maintained parallel to each other.

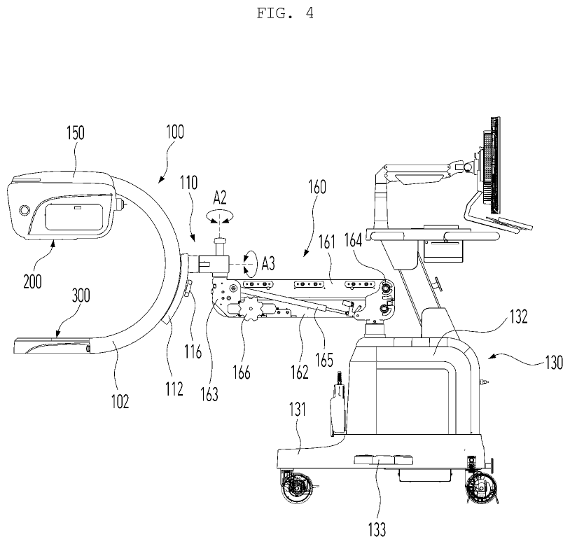

FIG. 4 is a side view of the internal structure of the arm part of the X-ray imaging apparatus of FIG. 1.

The arm part 160 according to this embodiment includes an upper link 161 and a lower link 162 as the pair of links that are parallel to each other and have the same length. The upper link 161 and the lower link 162 have the rear ends thereof hinge-coupled to a rear axis member 164 connected to the above-described first axis A1 (see FIG. 1), and the front ends thereof hinge-coupled to a front axis member 163 connected to the above-described second axis A2 (see FIGS. 1 and 3). In addition, the arm part 160 includes a gas spring 165 connecting the front axis member 163 and the lower link 162, and may further include a fixing knob 166 that locks the gas spring and fixes the posture of the arm part 160. The length between both ends of the gas spring 165 changes when the angle of the arm part 160 with respect to the ground changes, and the gas spring 165 is responsible for fixing or unfixing the posture of the arm part along with a damping function to ensure smooth and stable operation.

In this embodiment, the gas spring 165 is disposed between the upper part of the front axis member 163 and a portion of the rear side of the lower link 162, but is not limited thereto. As another example, the gas spring 165 may be disposed in various locations, such as between the lower part of the front axis member and the rear side of the upper link, or between the lower part of the rear axis member and the front side of the upper link.

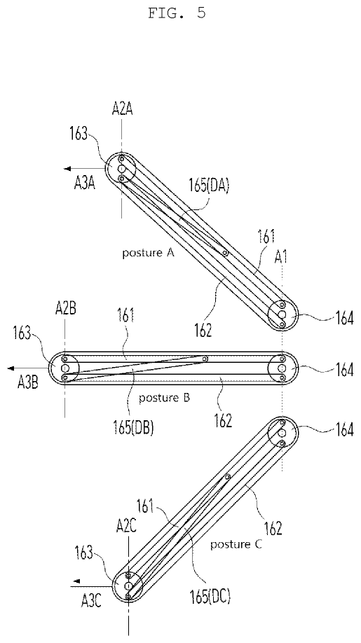

FIG. 5 schematically shows the operating principle of the arm part of the X-ray imaging apparatus according to an embodiment of the present disclosure.

FIG. 5 shows the configuration of the arm part according to this embodiment in an easy-to-understand manner through the relationship between the components constituting the arm part when the above-mentioned arm part 160 (see FIG. 4) is in three postures (A posture, B posture, and C posture) that are horizontal or inclined upward/downward with respect to the ground. As described above, the arm part according to this embodiment includes the upper link 161 and the lower link 162 that have the same length and are parallel to each other, the front axis member 163 and the rear axis member 164 hinge-coupled to each link at the front and rear ends of the upper and lower links 161 and 162, and the gas spring 165. In this case, the gas spring 165 is connected to the lower part of the front axis member 163 and a portion of the rear side of the upper link 161, but the connection position thereof is not limited as discussed above.

Meanwhile, the distance between the axes of the two connection points where the front ends of the upper and lower links 161 and 162 are respectively hinge-coupled in the front axis member 163 is equal to the distance between the axes of the two connection points where the rear ends of the upper and lower links 161 and 162 are respectively hinge-coupled in the rear axis member 164. Thus, the arm part according to this embodiment has a parallelogram structure in which two opposing sides, such as the upper and lower links 161 and 162 and the front and rear axis members 163 and 164, have the same length.

As a result, even if the inclination angle of the arm part with respect to the ground varies, such as the shown A posture, B posture, and C posture, in the front axis member 163 and the rear axis member 164, the extension line connecting the two connection points respectively hinge-coupled to the front ends of the upper and lower links 161 and 162 and the extension line connecting the two connection points respectively hinge-coupled to the rear ends of the upper and lower links 161 and 162 are always parallel to each other. In other words, when the extension line connecting the two connection points in the rear axis member 164 faces the aforementioned A1 axis direction, the extension line connecting the two connection points in the front axis member 163 points toward A2A, A2B, and A2C axes parallel to the first axis A1 in posture A, posture B, and posture C, respectively. As a result, directions of the A3A, A3B, and A3C axes, which are the normal directions to the A2A, A2B, and A2C axes, respectively, are also parallel to each other and always face horizontal to the ground.

However, what changes in the A posture, B posture, and C posture is the length of the gas spring 165, and lengths DA, DB, and DC of the gas spring in each posture have the relationship DA<DB<DC. The order of the size relationship between gas spring lengths may vary depending on the gas spring installation location.

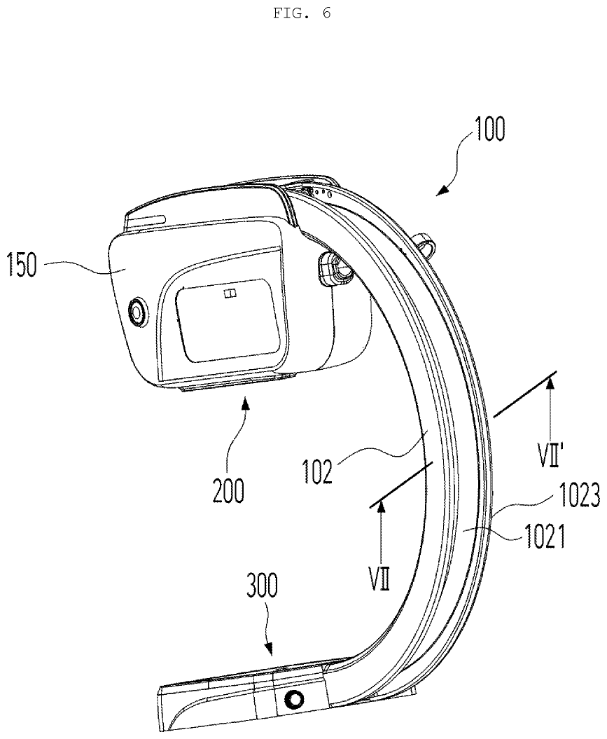

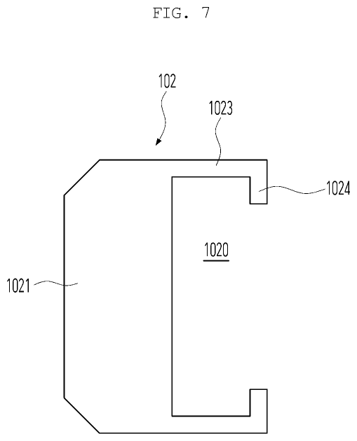

FIG. 6 is a view of the C-arm part according to an embodiment of the present disclosure as seen from the C-shaped arm side. FIG. 7 shows the cross-section (VII-VII′) profile of the C-shaped arm of FIG. 6.

Referring to FIGS. 6 and 7, the C-shaped arm 102, on which the generator 200 and the detector 300 are installed at opposite ends thereof, may be made of a curved beam with a predetermined cross-sectional profile extending in the length direction. The C-shaped arm 102 arranges a receiving space 1020 created by both side walls 1023 on the outside of a curved body 1021 forming a C-shaped curved surface, thereby reducing the load thereof and increasing the bending rigidity in the length direction. In addition, a portion of the C-arm supporter 110 (see FIGS. 1 to 4) and the C-shaped arm 102 may be relatively slidably accommodated in the receiving space 1020. At the outer end of the side wall 1023, an outer end protrusion 1024 protruding in a direction surrounding the receiving space 1020 to support a structure that moves relative to the C-shaped arm 102 along the receiving space 1020.

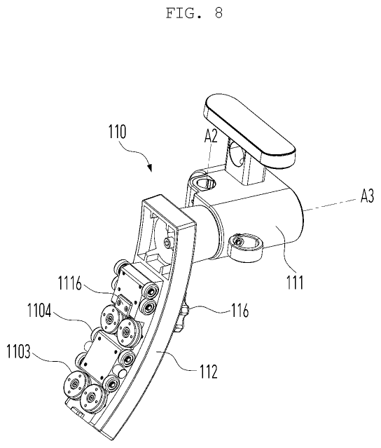

FIG. 8 shows the configuration of a C-arm supporter connected to the C-shaped arm of FIG. 6.

According to this embodiment, the C-arm supporter 110 may include: a slide connection part 112 that corresponds to a portion of the outer side of the C-shaped arm 102 and is slidably connected to the C-shaped arm 102; and a two-axes rotation connection part 1101 connected to the front axis member 163 of the above-described arm part 160 (hereinafter, see FIG. 3) to allow the slide connection part 112 to rotate about the above-described second axis A2 and third axis A3. The second axis A2 and the third axis A3 are orthogonal to each other, and the two-axes rotation connection part 1101 allows the C-shaped arm 102 to rotate around an arbitrary direction on the xy plane horizontal to the ground.

Referring to the above-mentioned FIGS. 6 and 7 together, the slide connection part 112 guides the sliding between the C-arm supporter 110 and the C-shaped arm 102 by using a plurality of rollers 1103 and 1104 accommodated in the receiving space 1020 formed on the outside of the C-shaped arm 102. The rollers 1103 and 1104 may include a side roller 1103 corresponding to the above-described side wall 1023, and a curved roller 1104 corresponding to the above-described curved body 1021 and the outer end protrusion 1024 of the side wall 1023.

Meanwhile, the slide connection part 112 may be provided with a C-arm fixing knob 116 that fixes the slide operation of the C-shaped arm 102 with respect to the slide connection part 112 in a stopped state. The C-arm fixing knob 116 is screwed from the outside to the inside of the slide connection part 112, and a brake pad 1116 may be disposed on the inner end of the screw. When a user turns the C-arm fixing knob 116, the brake pad 1116 is pressed toward the curved body 1021 of the C-shaped arm 102 by the screw connected to the inside, so that the slide connection part 112 and the C-shaped arm 102 are fixed to each other in a stationary state.

What is claimed is:

1. An X-ray imaging apparatus comprising:
a main body part configured to include a movable mobile base;
an arm part connected to the main body part through a first connection part and capable of rotating up, down, left and right based on the first connection part;
a C-arm supporter connected to the arm part through a second connection part and capable of rotating left and right based on the second connection part; and
a C-arm part configured to include a C-shaped arm connected to the C-arm supporter and capable of rotating with respect to the C-arm support, and an X-ray generator and an X-ray detector respectively disposed at opposite ends of the C-shaped arm,
wherein the first connection part and the second connection part remain parallel to each other with respect to the up-down, left-right rotation of the arm part.

2. The apparatus of claim 1, wherein the first and second connecting parts each comprise first and second axes,
wherein the arm part comprises:
upper and lower links parallel to each other;
a rear axis member connected to the first axis and to which rear ends of the upper and lower links are hinge-coupled; and
a front axis member connected to the second axis and to which front ends of the upper and lower links are hinge-coupled.

3. The apparatus of claim 2, further comprising:
a gas spring configured to connect the front axis member and the lower link or the rear axis member and the upper link.

4. The apparatus of claim 1, wherein the C-arm supporter is configured to be connected to the C-arm Part through a third axis,
wherein the C-arm supporter further comprises:
a slide connection part connected through the third axis,
wherein the C-shaped arm is connected to the slide connection part so as to slide along the slide connection part.

5. The apparatus of claim 4, wherein the slide connection part further comprises a C-arm fixing knob configured to screw through the slide connection part and secure a position of the C-shaped arm relative to the slide connection part.

6. The apparatus of claim 4, wherein the C-shaped arm comprises a receiving space provided along a length direction to accommodate the slide connection part, and
the slide connection part comprises a plurality of rollers rotating along the receiving space within the receiving space.
